(19) United States
(12) Patent Application Publication
(10) Pub. No.: US 2022/0347409 A1
(43) Pub. Date: Nov. 3, 2022

(54) DEVICE FOR INHALATION-SYNCHRONISED DISPENSING OF A FLUID PRODUCT

(71) Applicant: APTAR FRANCE SAS, LE NEUBOURG (FR)

(72) Inventor: David FABIEN, CORSEUL (FR)

(73) Assignee: APTAR FRANCE SAS, LE NEUBOURG (FR)

(21) Appl. No.: 17/296,720

(22) PCT Filed: Nov. 29, 2019
(86) PCT No.: PCT/FR2019/052837

§ 371 Date: May 25, 2021

(30) Foreign Application Priority Data
Nov. 30, 2018 (FR) FR1872152

(51) Int. Cl. A61M 15/00

(52) U.S. Cl. CPC A61M 15/0095; A61M 2205/0205; A61M 2205/276; A61M 2205/7545

(57) ABSTRACT

An inhalation-synchronized fluid product dispenser device having a body (10) provided with a mouthpiece (400), a product reservoir (100) containing fluid product, and a dispenser mechanism (200). The device has a blocking mechanism (500; 600) for the dispenser mechanism and an inhalation-controlled trigger system having an inhalation-sensitive member (60) deformable and/or movable by inhalation, the inhalation-sensitive member co-operating with the blocking mechanism so that when deformed and/or moved, it enables the dispenser mechanism to be actuated. The device has a protective element (1) the trigger system, the protective element disposed between the mouthpiece and the inhalation-sensitive member and made of a porous material that allows air to pass through it but blocks the passage of water, dust and foreign bodies.

The present invention relates to a fluid product dispenser device in which dispensing is synchronized with inhaling, and more particularly it relates to an inhaler device of the aerosol type synchronized with inhaling.

Breath actuated inhaler (BAI) devices are well known in the state of the art. The main advantage of this type of device is that the dispensing of product is synchronized with the patient inhaling, so as to guarantee that the product is properly dispensed into the airways. Thus, in the field of aerosol devices, i.e. devices in which the product is dispensed by means of a propellant gas, as well as in the field of dry-powder inhalers, in which the product is dispensed by means of the user inhaling, numerous types of inhalation-controlled trigger device have been proposed. However, these devices present the drawback of presenting risks of contamination, for example bacterial and/or microbial contamination, in the BAI system. One solution is to wash said BAI system after each use, but many devices are not suitable for this solution, and standing water in the device is also a source of potential contamination.

Documents WO2017178764, WO2016030521, WO0224265, US2018228989, and WO9209323 describe prior-art devices.

An object of the present invention is to provide an inhalation-synchronized fluid product dispenser device that does not have the above-mentioned drawbacks.

Another object of the present invention is to provide an inhalation-synchronized fluid product dispenser device that improves operational reliability by guaranteeing effective actuation on each inhalation. Another object of the present invention is to provide an inhalation-synchronized fluid product dispenser device that minimizes the risks of contamination, for example bacterial and/or microbial, of the product that is dispensed on each actuation.

Another object of the present invention is to provide an inhalation-synchronized fluid product dispenser device that can be washed after each use.

Another object of the present invention is to provide an inhalation-synchronized fluid product dispenser device that is simple and inexpensive to manufacture and to assemble.

The present invention thus relates an inhalation-synchronized fluid product dispenser device comprising a body provided with a mouthpiece, at least one product reservoir containing fluid product, and dispenser means for dispensing a dose of fluid product on each actuation, said device comprising blocking means for said dispenser means, said device comprising an inhalation-controlled trigger system comprising an inhalation-sensitive member that is deformable and/or movable under the effect of inhaling, said inhalation-sensitive member co-operating with said blocking means so that, when said inhalation-sensitive member is deformed and/or moved under the effect of inhaling, it enables said dispenser means to be actuated, said device comprising a protective element of said inhalation-controlled trigger system, said protective element being disposed between said mouthpiece and said inhalation-sensitive member, said protective element being made of a porous material that allows air to pass through it but blocks the passage of water, dust and foreign bodies.

Advantageously, said protective element is made of an open-pore sintered material.

Advantageously, said open-pore sintered material comprises a material of the polyolefin type, such as polyethylene (PE), polypropylene (PP) or a mixture thereof, and/or of the polytetrafluoroethylene (PTFE) type.

Advantageously, said protective element is subjected to an antibacterial treatment.

Advantageously, said product reservoir containing a fluid product and a propellant gas is mounted to slide axially relative to said body, a metering valve including a valve member being assembled on said reservoir for selectively dispensing the fluid product.

Advantageously, the device comprises a blocking element that is movable and/or deformable between a blocking position in which said metering valve cannot be actuated, and an actuation position in which said metering valve can be actuated, a trigger element that is movable and/or deformable between a locking position in which it blocks said blocking element in its blocking position, and a release position in which it does not block said blocking element, and said inhalation-sensitive member co-operating with said trigger element, so that when said inhalation-sensitive member is deformed and/or moved, it moves and/or deforms said trigger element towards its release position, thereby making it possible to move and/or deform said blocking element from its blocking position towards its actuation position.

Advantageously, an actuator member is mounted to move axially, in particular slidingly, in said body between a rest position and a primed position, a spring being arranged between said actuator member and said reservoir or an element integral with said reservoir, so that when said actuator member moves towards its primed position, said spring is compressed, so as to transmit an axial force to said reservoir.

Advantageously, a laterally-actuated pusher is mounted to move in pivoting and/or in translation on said body between a rest position and a working position, the movement of said laterally-actuated pusher towards its working position moving said actuator member axially towards its primed position.

Advantageously, said inhalation-sensitive member includes a deformable membrane that defines a deformable air chamber, said deformable membrane being fastened to said trigger element, said deformable membrane being deformed during inhaling.

These and other characteristics and advantages appear more clearly from the following detailed description, given by way of non-limiting example, and with reference to the accompanying drawings, in which:

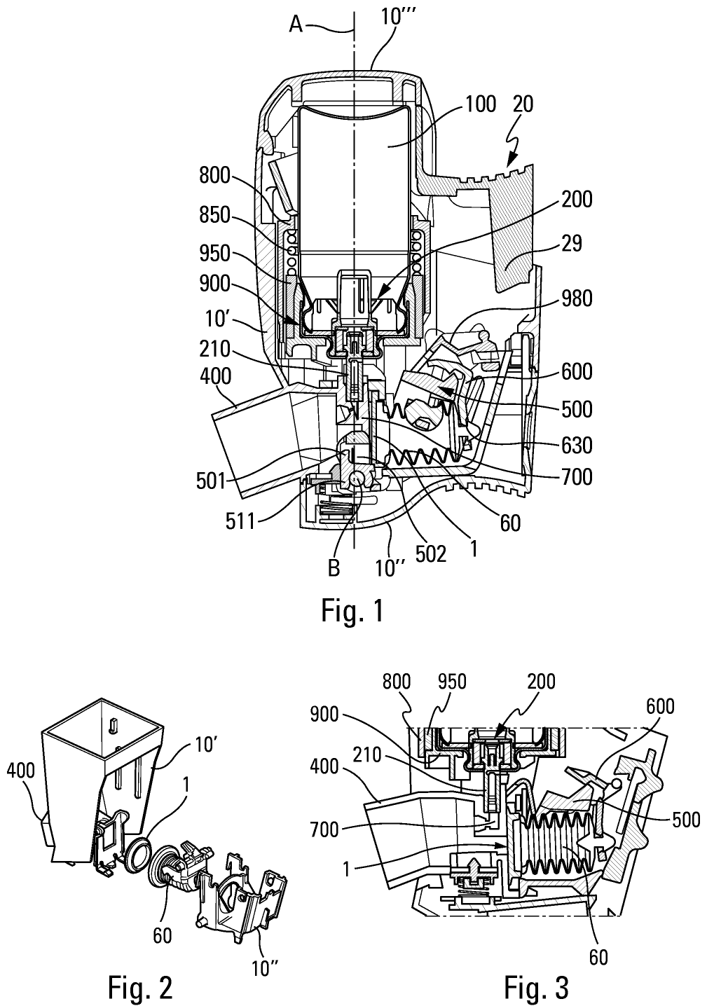

FIG. 1 is a diagrammatic sectional view of a fluid product dispenser device, in its rest position, according to an advantageous embodiment,

FIG. 2 is an exploded diagrammatic and fragmentary perspective view of a portion of the FIG. 1 device,

FIG. 3 is a cut away diagrammatic and fragmentary perspective view of a portion of the FIG. 1 device,

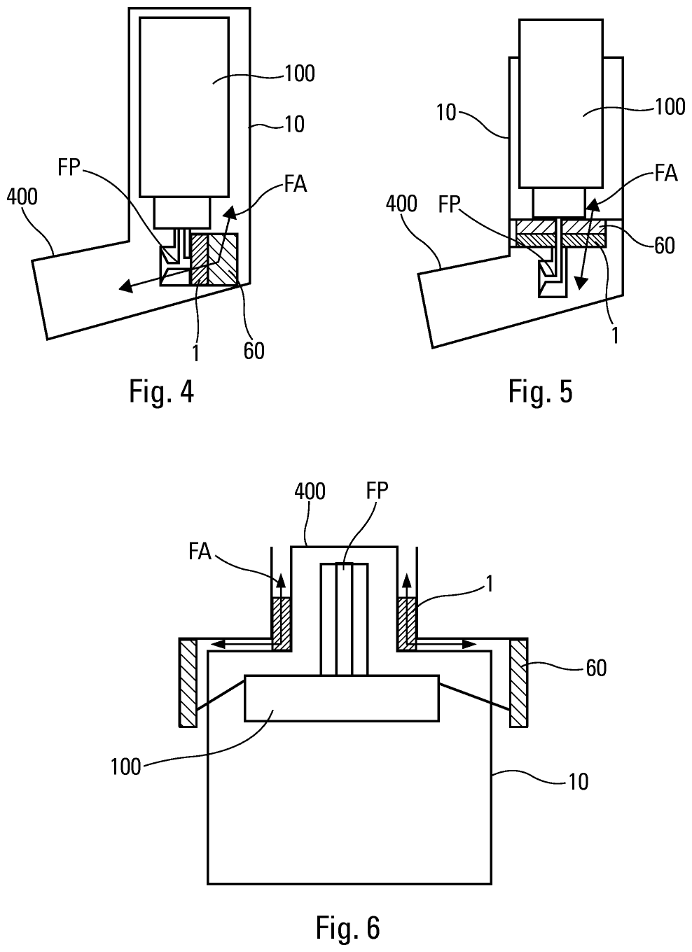

FIG. 4 is a very diagrammatic view of a fluid product dispenser device, according to an advantageous embodiment,

FIG. 5 a view similar to the view in FIG. 4, showing another advantageous embodiment, and

FIG. 6 a view similar to the views in FIGS. 4 and 5, showing still another advantageous embodiment.

In the description, the terms “top” and “bottom” refer to the upright position of the device shown in FIG. 1. The terms “axial” and “radial”, unless specified otherwise, are relative to the vertical central axis A of the valve shown in FIG. 1. The terms “proximal” and “distal” are relative to the mouthpiece.

The figures show advantageous embodiments of the invention, but it is understood that one or more of the component parts described below could be made in some other way, while providing functions that are similar or identical.

Only the embodiment in FIGS. 1 to 3 is shown and described below in detail, but it is understood that the present invention is not limited to this particular embodiment, and that any type of dispenser device, with or without propellant gas, and any type of BAI system are potentially adaptable to the present invention.

In known manner, an inhalation-synchronized fluid product dispenser device comprises a body 10 provided with a mouthpiece 400, at least one product reservoir 100 containing fluid product, and dispenser means 200 for dispensing a dose of fluid product on each actuation. The device includes blocking means 500, 600 for blocking said dispenser means 200, and an inhalation-controlled trigger system. The latter comprises an inhalation-sensitive member 60 that is deformable and/or movable under the effect of inhaling, which co-operates with the blocking means so that when said inhalation-sensitive member 60 is deformed and/or moved under the effect of inhaling, it enables said dispenser means 200 to be actuated.

According to the invention, the inhalation-controlled trigger system comprises a protective element 1 disposed between the mouthpiece 400 and the inhalation-sensitive member 60, said protective element 1 being made of a porous material allowing air to pass but blocking the passage of water, dust and foreign bodies.

Advantageously, the protective element 1 is a membrane made of an open-pore sintered material. This type of material makes it possible to disturb the flow of inhalation air as little as possible and thereby limits the increase in resistance of the system, while making it impossible for water to pass through it.

This material may comprise a material of the polyolefin type, such as polyethylene (PE), polypropylene (PP) or a mixture thereof, and/or of the polytetrafluoroethylene (PTFE) type. These materials have a naturally hydrophobic behaviour and pick up little or no moisture, which is consistent with avoiding any stagnation of liquid to avoid bacterial and/or microbial development.

Possible materials include Porex®, or Tyvek® from Dupont de Nemours. More generally, it is possible to envisage any material made from non-woven high-density polyethylene (HDPE) fiber. Here again, this type of material allows the passage of air with a controlled flow rate by offering the expected protection. Other materials include Sabeu Trak-echt®, which forms microperforated filters made of polymers with controlled porosity, or Millipore®, AP series, borosilicate based filter and acrylic binder.

Advantageously, the protective element 1 is subjected to an antibacterial treatment.

FIGS. 4 to 6 show in very diagrammatic manner various devices to which the present invention may apply. Thus, FIG. 4 illustrates a device as described below with reference to FIGS. 1 to 3, in which the porous protective element 1 is in fluid communication with the air flow FA and the product flow FP.

FIG. 5 illustrates a device in which the porous protective element 1 is in fluid communication with the flow of air FA but not with the flow of product FP.

Finally, FIG. 6 illustrates a device of the dry-powder inhaler type, in which the porous protective element 1 is in fluid communication with the air flow FA but not with the product flow FP.

In any case, the porous protective element 1 protects the inhalation-sensitive member 60. This protection makes it possible, among other advantages, to keep all the elements arranged behind this porous protection element 1 clean. This makes it possible in particular to clean and prevents bacterial and/or microbial proliferation.

An advantageous embodiment will be described below in greater detail with reference to FIGS. 1 to 3.

In this embodiment, the device includes a body 10 provided with a mouthpiece 400.

The body 10 may be made as a single piece or out of a plurality of parts that are assembled together. In the example shown, the body 10 comprises three portions, a central portion 10′, a bottom portion 10″ and a top portion 10′″. In the description below, the body is designated, in overall manner, by the numerical reference 10.

The mouthpiece 400 defines a dispenser orifice through which the user inhales while the device is being used. The mouthpiece 400 may be made integrally with the body 10. In the embodiments shown in the drawings, it is formed on the bottom body portion 10″. A removable protective cap (not shown) may be provided on said mouthpiece 400, in particular while it is being stored, that the user removes before use.

The body 10 contains a reservoir 100 that contains the product to be dispensed and a propellant gas, such as a gas of the hydrofluoroalkane (HFA) type, a metering valve 200 being mounted on said reservoir 100 for selectively dispensing the product. The metering valve 200 comprises a valve body, and a valve member 210 that, during actuation, is axially movable relative to said valve body, and thus relative to said reservoir 100. This metering valve 200 can be of any appropriate type. It is fastened to the reservoir 100 via a fastener element, preferably a crimped cap, preferably with a neck gasket interposed therebetween.

Advantageously, during actuation, the valve member 210 is stationary relative to the body 10, and it is the reservoir 100 that is moved axially relative to the body 10 between a distal position, which is the rest position, and a proximal position, which is the actuation position.

The outlet orifice of the valve member 210 of said metering valve 200 is connected via a channel to said mouthpiece 400 through which the user inhales the product to be dispensed. In known manner, said valve member 210 is received in a valve well 700 that defines said channel, at least in part.

The device includes a ring 900 that is advantageously fastened around said fastener element, e.g. by snap-fastening by means of snap-in tabs. Advantageously, a hoop 950 is sleeved around said ring 900, so as to hold said snap-in tabs in their snap-fitted position.

An actuator member 800 is advantageously assembled around the reservoir 100. The actuator member 800 is arranged in the body 10 around the reservoir 100, with a spring 850 arranged between its bottom and the reservoir 100 or an element that is secured to said reservoir 100, such as the ring 900 or the hoop 950. The actuator member 800 is axially movable, in particular slidingly, relative to said reservoir 100 between a rest position and a primed position. Thus, when the user wishes to actuate the metering valve 200, the user presses on said actuator member 800. This moves it towards its primed position and thus compresses said spring 850, which thus transmits an axial force F to said reservoir 100, in particular via said hoop 950, in the embodiment shown. The axial force F is substantially the same on each actuation. While the user continues to press on said actuator member 800, said spring 850 is compressed and bias said reservoir 100 axially towards its actuated position.

A laterally-actuated pusher 20 is advantageously mounted to move in pivoting and/or in translation on the body 10. When moved from its rest position shown in particular in FIG. 1, to its working position, the pusher 20 moves said actuator member 800 axially so as to compress the spring 850.

Advantageously, the pusher 20 is movable both in pivoting and in translation. This makes it possible to reduce the force required from the user, while remaining compact. This reduction makes it possible to actuate the valve 200 with a force that is smaller than the force that would be required if the user had to press axially on the bottom of the reservoir 100. In particular, in the embodiment shown, the force required to actuate the valve 200 axially is typically in the range 40-45N (depending on the stiffness of the spring 850), while the force required to actuate the laterally-actuated pusher 20 is only about 15 N. In this embodiment, the reduction is thus about a factor of three. It is possible to increase this ratio further, in particular by acting on the shapes of the various parts.

While the user continues to press on said pusher, said spring 850 is compressed and bias said reservoir 100 axially towards its actuation position.

After each actuation, when the user releases its pressure on the pusher 20, which occurs naturally, said pusher returns automatically towards its rest position under the effect of the spring 850. After the metering valve 200 has been actuated, this makes it possible to avoid the risk of said metering valve remaining in its actuated position, which could cause the valve chamber to fill with air and the following dose to be incomplete, or it could cause the valve to leak. This is one of the problems that currently exist with devices that are currently on the market.

The device includes a blocking element 500 that is movable and/or deformable between a blocking position in which said metering valve 200 cannot be actuated, and an actuation position in which said metering valve 200 can be actuated. In the rest position, said blocking element 500 is in the blocking position, and it is the user inhaling through the mouthpiece 400 that moves and/or deforms said blocking element 500 towards its actuation position. In other words, so long as the user does not inhale, it is impossible to actuate the metering valve 200, and it is only when the user inhales that said metering valve 200 can be actuated, by moving the reservoir 100 axially in the body 10.

As described in greater detail below, the blocking element 500, in its blocking position, prevents the reservoir 100 from moving axially in the body 10. During inhaling, the blocking element 500 is moved and/or deformed so that it no longer prevents the reservoir 100 from moving axially in the body 10. Thus, after inhaling, such axial movement of the reservoir 100 causes the metering valve 200 to be actuated and a dose of product to be dispensed, synchronously with the inhaling. Thus, in the absence of inhaling, there is no risk of an dose of active product being lost by accidental or incomplete actuation during which the user does not inhale. Actuating the valve 200 and expelling a dose of fluid product are thus possible only when the user inhales and simultaneously actuates the actuator pusher 20. Alternatively, it is also possible to envisage that the user presses axially, directly on the bottom of the reservoir, or it is possible to use an automatic actuator system that would apply the axial pressure on the reservoir independently of the user.

The device includes a trigger system that is controlled by the user inhaling, and that is for moving and/or deforming said blocking element 500 from its blocking position towards its actuation position, when the user inhales through the mouthpiece 400.

The trigger system includes an inhalation-sensitive member 60 that is deformable and/or movable under the effect of inhaling, the inhalation-sensitive member 60 being adapted, when it is deformed and/or moved, to make it possible to move and/or deform said blocking element 500 from its blocking position towards its actuation position.

According to the invention, said inhalation-sensitive member 60 is provided, on the side connected to the mouthpiece 400, with a porous protective element 1, advantageously made in the form of a membrane.

As described in greater detail below, the inhalation-sensitive member may be made in the form of a deformable air chamber 60, e.g. a bellows or a deformable pouch.

The inhalation-controlled trigger system is thereby not situated in the user's suction flow but is formed by a specific chamber, namely the air chamber 60. This differs from systems that operate by means of a flap that moves/deforms in the suction flow, in which systems, after triggering, the user sucks in the air that exists on each side of the flap. In this embodiment, the system operates under reduced pressure and the user sucks in only the small volume of air that was inside the air chamber 60 before it deformed. The system according to the invention is thus much more stable and effective.

The blocking element 500 is advantageously mounted to pivot about an axis B on the body 10, between a blocking position and an actuation position. In the embodiment shown, said axis B may be formed by projections that are provided on a bottom surface of the body 10, the blocking element 500 including complementary profiles 511 that are adapted to pivot on said projections. Other embodiments are also possible.

The blocking element 500 includes at least one, preferably two, blocking extensions 501, each of which co-operates in the blocking position with a respective axial projection of said ring 900 that is secured to the reservoir 100.

When the blocking element 500 moves towards its actuation position, in particular by pivoting about the axis B, each blocking extension 501 moves out of contact with the respective axial projection of said ring 900. In particular, adjacent to each blocking extension 501, said blocking element 500 includes an axial recess 502 in which the respective axial projection of said ring 900 can slide axially, thereby enabling said reservoir 100 to slide axially in said body 10, causing the valve 200 to be actuated and a dose of fluid product to be dispensed.

The blocking element 500 is held in its blocking position by a trigger element 600. The trigger element 600 is advantageously mounted to pivot about an axis on the body 10, between a locking position in which it blocks said blocking element 500 in its blocking position, and a release position in which it no longer blocks said blocking element 500.

Advantageously, the axes of the blocking element 500 and of the trigger element 600 are parallel.

The blocking system of the present invention thus includes two stages: a first stage formed by the latch between the blocking element 500 and the trigger element 600, and a second stage formed by the blocking between the blocking element 500 and the reservoir 100, via the ring 900.

The blocking system makes it possible to unlock a large force (typically about 40 N to 45 N) by means of a small force generated by inhaling. The blocking element 500 stops the reservoir 100 from moving in translation when it is subjected to a force F (e.g. of 45 N) by means of the user pressing on the actuator member 800, preferably via the actuator pusher 20. The blocking element 500 interacts with the trigger element 600, and it is both blocked and released by said trigger element. The movement of said trigger element 600 is controlled by inhaling.

The shape of the blocking system enables very large amplification (locked force/unlocked force), typically of about 100.

The unlocking force generated by inhaling is applied to the trigger element 600 by the deformable membrane 60, preferably at a point 630 at a distance from the pivot axis of the trigger element 600.

By means of this force system of the latch, the force necessary to cause the trigger element 600 to pivot is very small and may be generated by the deformable membrane 60 that makes it possible to transform the reduced pressure generated by inhaling into unlocking force.

In the embodiment shown in the FIG. 1, the inhalation-sensitive member 60 is made in the form of a deformable air chamber. Advantageously, the air chamber comprises a deformable membrane that is connected firstly to said bottom body portion 10′ and secondly to said trigger element 600. Advantageously, the inhalation-sensitive member 60 is in the form of a bellows and forms a substantially airtight chamber. Other forms are possible, in particular a mere pouch or diaphragm. A lug may fasten the inhalation-sensitive member 60 to an orifice or edge 630 of said trigger element 600.

During inhaling, the inhalation-sensitive member 60 deforms and/or contracts under the effect of the reduced pressure generated by inhaling, causing the trigger element 600 to move from its locked position towards its release position. This makes it possible to open the latch defined between the blocking element 500 and the trigger element 600, and thus to move said blocking element 500 from its blocking position towards its actuation position.

The valve 200 is thus actuated only at the moment of inhaling, such that the dose of fluid product is expelled out of the dispenser orifice simultaneously with inhaling.

Advantageously, the device includes a blocking member 980 that is movable and/or deformable between a blocking position and a non-blocking position. In its blocking position, the blocking member 980 co-operates with the trigger element 600 so as to prevent it from moving towards its release position. The laterally-actuated pusher 20 advantageously includes a projection 29 that co-operates with said blocking member 980 when said laterally-actuated pusher 20 is moved towards its working position. This moves and/or deforms said blocking member 980 towards its non-blocking position.

Thus, when the user inhales without having pressed axially on the reservoir 100, the latch is not unblocked, since the trigger element 600 cannot pivot. Since the air chamber 60 is substantially airtight, the user very quickly realizes that it is not possible to inhale correctly through the mouthpiece 400, which reminds the user that it is necessary to actuate the pusher 20 first before inhaling. When the user presses on the pusher 20, the blocking member 980 is moved into its non-blocking position. Inhaling thus causes the trigger element 600 to pivot, and thus causes the device to be actuated, as explained above.

Advantageously, the blocking member 980 includes a resilient element (not shown), such as a torsion spring, that resiliently bias said blocking member 980 towards its blocking position, so that when the user releases the actuator pusher 20, said blocking member 980 returns automatically into its blocking position. Advantageously, this also returns the trigger element 600 into its locking position, e.g. via an appropriate flexible blade.

When the user wishes to use the device, the user places the mouthpiece 400 in the mouth, and exerts axial pressure manually on the actuator pusher 20. The reservoir 100 is blocked and prevented from sliding axially in the body 10 by the blocking extensions 501 of the blocking element 500 that block the axial projections of the ring 900 axially. Simultaneously, the trigger element 600 is no longer blocked as a result of the movement of the blocking member 980.

When the user inhales through the mouthpiece 400, the inhalation-sensitive member 60 deforms, and this causes the trigger element 600 that is fastened to said deformable membrane 60 to pivot. The movement of the trigger element 600 releases the latch formed between the trigger element 600 and the blocking element 500. Under the effect of the axial force F transmitted by the reservoir 100, the blocking element 500 pivots enabling the reservoir 100 to slide axially in the body 10 towards its dispensing position, and the valve 200 thus to be actuated.

At the end of inhaling, when the user releases the pressure on the bottom of the reservoir 100, in particular by releasing the pressure on the pusher 20, said reservoir 100 rises axially in the body 10 towards its rest position under the effect of the return spring of the valve 200, and the valve member 210 of the metering valve simultaneously returns to its rest position, once again filling the valve chamber with a new dose of fluid product. The trigger element 600 is returned into its initial position, in particular by the springiness of the membrane 60 and/or the spring blade of the blocking member 980. The blocking element 500 returns into its blocking position, via a re-cocking extension of the blocking member 980.

The device is thus ready for another utilization.

It should be observed that the device could include an electronic dose counter, advantageously assembled in the body or in the pusher. In particular, the counter could detect the movements of the reservoir. Alternatively, the counter could be connected to a sensor, in particular a membrane sensor, that detects the dose of fluid product being dispensed, e.g. in the valve well. Such an electronic counter could be actuated in other ways, e.g. by detecting the movement of the valve member of the metering valve relative to the valve body.

The present invention applies, in particular, to treating asthma attacks or chronic obstructive pulmonary disease (COPD), by using formulations of the following types: salbutamol, aclidinium, formoterol, tiotropium, budesonide, fluticasone, indacaterol, glycopyrronium, salmeterol, umeclidinium bromide, vilanterol, olodaterol, or striverdi, or any combination of these formulations.

The present invention is described above with reference to advantageous embodiments and variants, but naturally any modification could be applied thereto by a person skilled in the art, without going beyond the ambit of the present invention, as defined by the accompanying claims.

1. inhalation-synchronized fluid product dispenser device comprising a body provided with a mouthpiece, at least one fluid reservoir containing fluid, and dispenser means for dispensing a dose of fluid on each actuation, said device comprising blocking means for said dispenser means, said device comprising an inhalation-controlled trigger system comprising an inhalation-sensitive member that is deformable and/or movable under the effect of inhaling, said inhalation-sensitive member co-operating with said blocking means so that, when said inhalation-sensitive member is deformed and/or moved under the effect of inhaling, it enables said dispenser means to be actuated, characterized in that said device comprising a protective element of said inhalation-controlled trigger system, said protective element being disposed between said mouthpiece and said inhalation-sensitive member, said protective element being made of a porous material that allows air to pass through it but blocks the passage of water, dust and foreign bodies.

2. The device according to claim 1, wherein said protective element is made of an open-pore sintered material.

3. The device according to claim 2, wherein said open-pore sintered material comprises a material of the polyolefin type, such as polyethylene, polypropylene or a mixture thereof, and/or of the polytetrafluoroethylene type.

4. The device according to claim 1, wherein said protective element is subjected to an antibacterial treatment.

5. The device according to claim 1, wherein said product reservoir containing a fluid product and a propellant gas is mounted to slide axially relative to said body, a metering valve, including a valve member, being assembled on said reservoir for selectively dispensing the fluid product.

6. The device according to claim 5, comprising a blocking element that is movable and/or deformable between a blocking position in which said metering valve cannot be actuated, and an actuation position in which said metering valve can be actuated, a trigger element that is movable and/or deformable between a locking position in which it blocks said blocking element in its blocking position, and a release position in which it does not block said blocking element, and said inhalation-sensitive member co-operating with said trigger element, so that when said inhalation-sensitive member is deformed and/or moved, it moves and/or deforms said trigger element towards its release position, thereby making it possible to move and/or deform said blocking element from its blocking position towards its actuation position.

7. The device according to claim 1, wherein an actuator member is mounted to move axially, in particular in sliding, in said body between a rest position and a primed position, a spring being arranged between said actuator member and said reservoir or an element that is secured to said reservoir, so that when said actuator member moves towards its primed position, said spring is compressed, so as to transmit an axial force to said reservoir.

8. The device according to claim 7, wherein a laterally-actuated pusher is mounted to move in pivoting and/or in translation on said body between a rest position and a working position, a movement of said laterally-actuated pusher towards its working position moving said actuator member axially towards its primed position.

9. The device according to claim 1, wherein said inhalation-sensitive member includes a deformable membrane that defines a deformable air chamber, said deformable membrane being fastened to said trigger element, said deformable membrane being deformed during inhaling.
